(19) United States
(12) Patent Application Publication
(10) Pub. No.: US 2003/0220512 A1
(43) Pub. Date: Nov. 27, 2003

(54) Novel transition metal complexes and their use in transition metal-catalysed reactions

(76) Inventor: Siegfried Blechert, Berlin (DE)

Correspondence Address: BAYER CHEMICALS CORPORATION, 100 BAYER ROAD, PITTSBURGH, PA, 15205, US

(21) Appl. No.: 10/439,159

(22) Filed: May 16, 2003

(30) Foreign Application Priority Data
May 17, 2002 (DE) 10222551.6

(51) Int. Cl. C07F 15/00; C07C 2/02

(52) U.S. Cl. 556/13; 556/136; 585/527

(57) ABSTRACT

The invention relates to novel transition metal complexes of the formula (I)

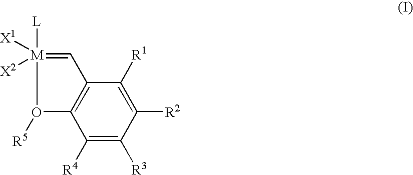

to processes for preparing these transition metal complexes, to intermediates for preparing them, and also to the use of the transition metal complexes as catalysts in organic reactions, particularly in metathesis reactions.

### BACKGROUND OF THE INVENTION

1. Field of the Invention

The invention relates to novel transition metal complexes of the formula (I), to processes for preparing these transition metal complexes, to intermediates for preparing them, and also to the use of the transition metal complexes as catalysts in organic reactions, particularly in metathesis reactions.

2. Brief Description of the Prior Art

Olefin metathesis constitutes an important synthetic method for C—C bond formation, since this reaction allows by-product-free olefins to be synthesized. This advantage is utilized not only in the field of preparative organic chemistry (ring-closing metathesis (RCM), ethenolysis, metathesis of acyclic olefins, cross-metathesis (CM)) but also in the field of polymer chemistry (ring-opening metathesis polymerizations (ROMP), alkyne polymerization, and acyclic diene metathesis polymerization (ADMET)).

For olefin metathesis, a multiplicity of catalyst systems is available. For instance, WO 99/51344 A1, WO 00/15339 A1 and WO 00/71554 A2 describe transition metal complexes which preferably bear ligands from the group of imidazol-2-ylidene, dihydroimidazol-2-ylidene and phosphine. The transition metal complexes mentioned are used as catalysts in olefin metathesis. A disadvantage of the catalysts described in the above-cited references is their low stability which manifests itself in very short catalyst on-stream times, which are highly disadvantageous, especially for industrial applications. After a high starting activity, the catalyst activity falls rapidly. In addition, the catalyst activity of these catalysts is strongly substrate-dependent.

Hoveyda et al., J. Am. Chem. Soc. 1999, 791-799 describe ruthenium complexes which, in addition to a phosphine ligand, have an alkoxybenzylidene ligand and are notable for higher stability in comparison to the systems known hitherto. One of the complexes described is suitable as a recyclable catalyst in metathesis reactions.

Gessler et al., Tetrahedron Lett. 41, 2000, 9973-9976 and Garber et al., J. Am. Chem. Soc. 122, 2000, 8168-8179 describe ruthenium complexes which, in addition to a dihydroimidazol-2-ylidene ligand, have an isopropoxybenzylidene ligand. The ruthenium complexes mentioned are used as catalysts in metathesis reactions, and, as mentioned for the above-described compound, can be removed from the reaction mixture and reused in a further metathesis reaction. A disadvantage of these reusable catalyst systems is their only moderate activities in comparison to the systems known hitherto.

In the German patent having the reference number 10137051, which was unpublished at the priority date of the present invention, complexes of the 8th transition group are described which, in addition to a dihydroimidazol-2-ylidene or an imidazol-2-ylidene ligand, have a substituted isopropoxybenzylidene ligand. The transition metal complexes of group 8 mentioned may likewise be used as catalysts in metathesis reactions, and have increased activity and also increased stability in comparison to the systems known hitherto. WO 02/14376 A2 describes in particular dendrimeric ruthenium complexes which have above-described ligands and can be more efficiently removed from the reaction products in catalytic reactions.

There is therefore a need for novel catalyst systems for olefin metathesis which are stable and air-stable and, in addition, exhibit high activities, and can be used as an alternative to the existing catalysts.

### SUMMARY OF THE INVENTION

Surprisingly, compounds of the following formula (I) have now been found to be useful as catalysts:

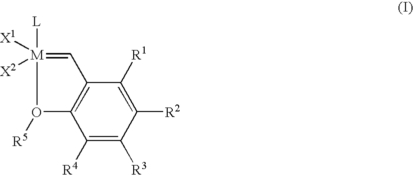

where

M is a transition metal of the 8th transition group of the Periodic Table,

X¹ and X² are the same or different and are each an anionic ligand,

R¹, R², R³ and R⁴ are the same or different, and are each hydrogen, with the proviso that at least one radical R¹ to R⁴ is different from hydrogen, or are each cyclic, straight-chain or branched alkyl radicals having 1 to 50 carbon atoms or aryl radicals having 6 to 30 carbon atoms, at least one hydrogen atom in the radicals mentioned, optionally being replaced by an alkyl group or a functional group, and at least one of the radicals R¹ to R⁴ is halogen, C₁-C₄-haloalkyl, C₁-C₄-alkoxy, C₆-C₁₀-aryloxy, cyano, C₁-C₄-alkoxy-carbonyl, C₆-C₁₀-aryloxycarbonyl or aliphatic or aromatic C₁-C₁₀-acyloxy, and/or

R¹ and R² or R² and R³ or R³ and R⁴ or R⁴ and R⁵ are part of a cyclic system which consists of a carbon framework having 3 to 20 carbon atoms, not including the carbon atoms in formula (I), at least one hydrogen atom in the radical mentioned, optionally being replaced by an alkyl group or a functional group, and/or at least one carbon atom of the cycle optionally being replaced by a heteroatom from the group of S, P, O and N, and

R⁵ is hydrogen or a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, and R² and R³ may not be part of a cyclic, aromatic system having 4 carbon atoms, not including the carbon atoms in formula (I), when R⁵ is at the same time methyl, and

L is a neutral two-electron donor from the group of amines, imines, phosphines, phosphites, stibines, arsines, CO, carbonyl compounds, nitrites, alcohols, thiols, ethers and thioethers.

### DETAILED DESCRIPTION OF THE INVENTION

The invention is described more fully hereunder with particular reference to its preferred embodiments.

The functional groups mentioned above and in the following are preferably radicals from the group of halogen, C₁-C₄-haloalkyl, C₁-C₄-alkoxy, C₁-C₆-aryloxy, cyano, C₁-C₄-alkoxycarbonyl, C₁-C₆-aryloxycarbonyl, aliphatic or aromatic C₁-C₆-acyloxy and sulphonic acid groups.

Areas of preference of the radicals present in the above-cited formulae are defined hereinbelow:

M is preferably ruthenium or osmium,

X¹ and X² are the same or different and are preferably each an anionic ligand from the group of halides, pseudohalides, hydroxides, alkoxides, carboxylates and sulphonates, the pseudohalides preferably being cyanide, thiocyanate, cyanate, isocyanate and isothiocyanate,

R¹, R², R³ and R⁴ are the same or different, and are preferably each hydrogen, with the proviso that at least one radical R¹ to R⁴ is different from hydrogen, or are each cyclic, straight-chain or branched alkyl radicals having 1 to 20 carbon atoms or aryl radicals having 6 to 20 carbon atoms, at least one hydrogen atom in the alkyl or aryl radicals mentioned optionally being replaced by an alkyl group or a functional group, and at least one of the radicals R¹ to R⁴ is preferably halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or

R¹, R² and R³ are preferably each hydrogen and R⁴ is preferably a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-alkoxy, C₁-C₄-haloalkyl, C₆-C10-aryloxy, cyano, C₁-C₄-alkoxycarbonyl, C₆-C₁₀-aryloxycarbonyl or aliphatic or aromatic C₁-C₁₀-acyloxy, or

R², R³ and R⁴ are preferably each hydrogen and R¹ is preferably a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or

R¹, R³ and R⁴ are preferably each hydrogen and R² is preferably a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or

R¹, R² and R⁴ are preferably each hydrogen and R³ is preferably a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or

R¹ and R⁴ are the same or different and are preferably each hydrogen or an aryl radical having 6 to 20 carbon atoms, at least one hydrogen atom in the aryl radical optionally being replaced by an alkyl group or a functional group, or are each halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy and R² and R³ are part of a cyclic aromatic system having 4 to 14 carbon atoms, not including the carbon atoms in formula (I) at least one hydrogen atom optionally being replaced by an alkyl group or a functional group, or

R¹ and R² are the same or different and are preferably each hydrogen or an aryl radical having 6 to 20 carbon atoms, at least one hydrogen atom in the aryl radical optionally being replaced by an alkyl group or a functional group, or are each halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy and R³ and R⁴ are part of a cyclic aromatic system having 4 to 14 carbon atoms, not including the carbon atoms in formula (I), at least one hydrogen atom optionally being replaced by an alkyl group or a functional group.

R⁵ is preferably a straight-chain or branched alkyl radical having 1 to 20 carbon atoms, and R² and R³ may not be part of a cyclic, aromatic system having 4 carbon atoms, not including the carbon atoms in formula (I), when R⁵ is at the same time methyl,

and L may be as defined above;

L is preferably a phosphine ligand PR⁷R⁸R⁹, with the proviso that R⁷, R⁸ and R⁹ maybe the same or different and are each cyclic, straight-chain or branched alkyl radicals having 1-10 carbon atoms or aryl radicals having 6 to 14 carbon atoms, at least one hydrogen atom in the alkyl or aryl radicals mentioned optionally being replaced by an alkyl group or a functional group.

M is more preferably ruthenium.

X¹ and X² are more preferably the same and are each an anionic ligand from the group of halides and pseudohalides, the pseudohalides preferably being cyanide, thiocyanate, cyanate and isocyanate.

R¹, R², R³ and R⁴ are the same or different and are more preferably each hydrogen, with the provision that at least one radical R¹ to R⁴ is different from hydrogen, or are each cyclic, straight-chain or branched alkyl radicals having 1 to 10 carbon atoms or aryl radicals having 6 to 14 carbon atoms, at, least one hydrogen atom in the alkyl or aryl radicals mentioned optionally being replaced by an alkyl group or a functional group, and at least one of the radicals R¹ to R⁴ is more preferably halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or

R¹, R² and R³ are each more preferably hydrogen and R⁴ is more preferably an aryl radical having 6 to 14 carbon atoms, at least one hydrogen atom in the aryl radical optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or

R¹ is more preferably hydrogen or halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy and R⁴ is more preferably hydrogen or an aryl radical having 6 to 14 carbon atoms, at least one hydrogen atom in the aryl radical optionally being replaced by an alkyl group or a functional group, preferably by C₁-C₄-alkyl or C₁-C₄-alkoxy, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, and R² and R³ are part of -a cyclic aromatic system having 4 to 8 carbon atoms; not including the carbon atoms in formula (I), and at least one hydrogen atom optionally being replaced by an alkyl group or a functional group, or

R¹ is particularly more preferably hydrogen or halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy and R² is more preferably hydrogen or an aryl radical having 6 to 14 carbon atoms, at least one hydrogen atom in the aryl radical optionally being replaced by an alkyl group or a functional group, preferably by C₁-C₄-alkyl or C₁-C₄-alkoxy, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, and R³ and R⁴ are part of a cyclic aromatic system having 4 to 8 carbon atoms, not including the carbon atoms in formula (I), and at least one hydrogen atom optionally being replaced by an alkyl group or a functional group.

R⁵ is more preferably a branched alkyl radical having 3 to 8 carbon atoms.

L is more preferably a phosphine PR⁷R⁸R⁹, with the proviso that R⁷, R⁸ and R⁹ are the same and are each cyclic, straight-chain or branched alkyl radicals having 1-10 carbon atoms or aryl radicals having 6 to 14 carbon atoms, at least one hydrogen atom in the alkyl or aryl radicals mentioned optionally being replaced by an alkyl group or a functional group.

M is most preferably ruthenium.

X¹ and X² are most preferably the same and are each halide, preferably chloride.

R² and R³ are most preferably the same and are each hydrogen and R¹ is hydrogen or a radical from the group of F, Cl, Br, trifluoromethyl, methoxy, ethoxy, isopropoxy, tert-butoxy, phenoxy, cyano, methoxycarbonyl, ethoxy-carbonyl, tert-butoxycarbonyl, acetoxy, propionyloxy and pivaloyloxy, and R⁴ is phenyl or naphthyl, at least one hydrogen atom optionally being replaced by an alkyl group or a functional group, preferably by C₁-C₄-alkyl or C₁-C₄-alkoxy, or is a radical from the group of F, Cl, Br, trifluoromethyl, methoxy, ethoxy, isopropoxy, tert-butoxy, phenoxy, cyano, methoxycarbonyl, ethoxycarbonyl, tert-butoxycarbonyl, acetoxy, propionyloxy and pivaloyloxy, or

R¹, R² and R³ are most preferably each hydrogen and R⁴ is most preferably a phenyl or naphthyl radical, at least one hydrogen atom optionally being replaced by an alkyl group or a functional group, preferably by C₁-C₄-alkyl or C₁-C₄-alkoxy, or is a radical from the group of F, Cl, Br, trifluoromethyl, methoxy, ethoxy, isopropoxy, tert-butoxy, phenoxy, cyano, methoxycarbonyl, ethoxycarbonyl, tert-butoxycarbonyl, acetoxy, propionyloxy and pivaloyloxy, or

R¹, R² and R⁴ are most preferably each hydrogen and R³ is most preferably a radical from the group of F, Cl, Br, trifluoromethyl, methoxy, ethoxy, isopropoxy, tert-butoxy, phenoxy, cyano, methoxycarbonyl, ethoxy-carbonyl, tert-butoxycarbonyl, acetoxy, propionyloxy and pivaloyloxy, or

R², R³ and R⁴ are most preferably each hydrogen and R² is most preferably a radical from the group of F, Cl, Br, trifluoromethyl, methoxy, ethoxy, isopropoxy, tert-butoxy, phenoxy, cyano, methoxycarbonyl, ethoxy-carbonyl, tert-butoxycarbonyl, acetoxy, propionyloxy and pivaloyloxy, or

R², R³ and R⁴ are most preferably each hydrogen and R¹ is most preferably a radical from the group of F, Cl, Br, trifluoromethyl, methoxy, ethoxy, carbonyl, tert-butoxycarbonyl, acetoxy, propionyloxy and pivaloyloxy, or

R¹ is most preferably hydrogen or a radical from the group of F, Cl, Br, trifluoromethyl, methoxy, ethoxy, isopropoxy, tert-butoxy, phenoxy, cyano, methoxycarbonyl, ethoxycarbonyl, tert-butoxycarbonyl, acetoxy, propionyloxy and pivaloyloxy and R⁴ is most preferably hydrogen or phenyl or naphthyl, at least one hydrogen atom optionally being replaced by an alkyl group or a functional group, preferably by C₁-C₄-alkyl or C₁-C₄-alkoxy, or is a radical from the group of F, Cl, Br, trifluoromethyl, methoxy, ethoxy, isopropoxy, tert-butoxy, phenoxy, cyano, methoxy-carbonyl, ethoxycarbonyl, tert-butoxycarbonyl, acetoxy, propionyloxy and pivaloyloxy and R² and R³ are most preferably part of a cyclic aromatic system having 4 to 8 carbon atoms, not including the carbon atoms in formula (I), and at least one hydrogen atom optionally being replaced by an alkyl group or a functional group, preferably by C₁-C₄-alkyl or C₁-C₄-alkoxy, or

R¹ is most preferably hydrogen or a radical from the group of F, Cl, Br, trifluoromethyl, methoxy, ethoxy, isopropoxy, tert-butoxy, phenoxy, cyano, methoxycarbonyl, ethoxycarbonyl, tert-butoxycarbonyl, acetoxy, propionyloxy and pivaloyloxy and R² is most preferably hydrogen or phenyl or naphthyl, at least one hydrogen atom optionally being replaced by an alkyl group or a functional group, preferably by C₁-C₄-alkyl or C₁-C₄-alkoxy, or is a radical from the group of F, Cl, Br, trifluoromethyl, methoxy, ethoxy, isopropoxy, tert-butoxy, phenoxy, cyano, methoxy-carbonyl, ethoxycarbonyl, tert-butoxycarbonyl, acetoxy, propionyloxy and pivaloyloxy and R³ and R⁴ are most preferably part of a cyclic aromatic system having 4 to 8 carbon atoms, not including the carbon atoms in formula (I), and at least one hydrogen atom optionally being replaced by an alkyl group or a functional group, preferably by C₁-C₄-alkyl or C₁-C₄-alkoxy.

R⁵ is most preferably a branched alkyl radical from the group of isopropyl, isobutyl, sec-butyl, tert-butyl, branched pentyl, branched hexyl.

L is most preferably a phosphine PR⁷R⁸R⁹, with the proviso that R⁷, R⁸ and R⁹ are the same and are each methyl, ethyl, cyclopentyl, cyclohexyl or phenyl.

Very particular preference is also given to the compounds of the formula (II) to (V)

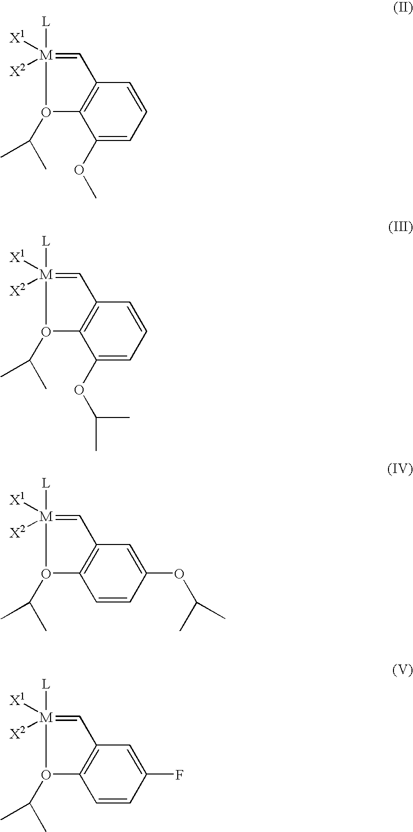

where

L is tricyclohexylphosphine,

X¹ and X²are each chloride and

M is ruthenium.

The above-cited radical definitions and illustrations cited in general or within areas of preference, i.e. also the particular areas and areas of preference also, may be combined with each other as desired. They apply correspondingly to the end products and also to the precursors and intermediates.

The above-mentioned functional groups are preferably radicals from the group of halogen, C₁-C₄-haloalkyl, C₁-C₄-alkoxy, C₁-C₆-aryloxy, cyano, C₁-C₄-alkoxy-carbonyl, C₁-C₆-aryloxycarbonyl, aliphatic or aromatic C₁-C₆-acyloxy and sulphonic acid groups.

The above-mentioned alkyl groups, by which hydrogen atoms in R¹ to R⁵ optionally being replaced, are preferably radicals from the group of C₁-C₄-alkyl, such as methyl, ethyl, n-propyl, isopropyl, n-butyl, isobutyl, sec-butyl or tert-butyl.

In addition to air stability and tolerance towards functional groups, the compounds of the formula (I) according to the invention exhibit distinctly higher activities in metathesis reactions in comparison to the existing systems, for example the systems described in Tetrahedron Lett. 41, 2000, 9973-9976 and in J. Am. Chem. Soc. 122, 2000, 8168-8179, which is demonstrated in the present application with the aid of examples. The compounds of the formula (I) according to the invention are equally suitable for ring-closing metatheses, ring-opening metatheses, cross-metatheses and ring-opening metathesis polymerizations.

The compounds of the formula (I) according to the invention are preferably prepared by exchange reaction of the phosphine ligand PZ₃ in compounds of the formula (VI) by ligands of the formula (VII)

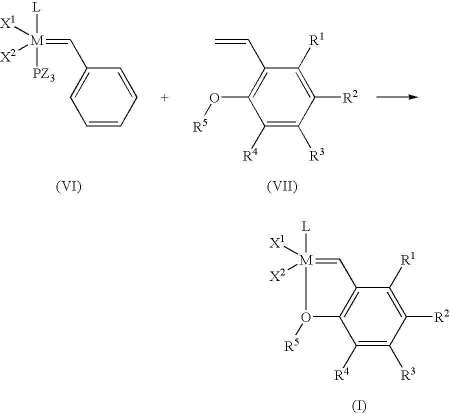

where

L has one of the above definitions,

M, R¹-R⁵, X¹ and X² each have one of the above definitions and

PZ₃ is a phosphine ligand, preferably trimethylphosphine, triethylphosphine, tricyclopentylphosphine, tricyclohexylphosphine or triphenylphosphine.

The compounds of the formula (I) according to the invention are preferably prepared from compounds of the formula (VI) in a solvent, more preferably in toluene, benzene, tetrahydrofuran or dichloromethane, most preferably in dichloromethane. The reaction preferably takes place in the presence of compounds which are capable of scavenging phosphines, more preferably in the presence of CuCl₂ and CuCl, most preferably in the presence of CuCl. Preference is given to working in the presence of equimolar amounts or of an excess of phosphine scavenger, based on compounds of the formula (VI). When CuCl is used as the phosphine scavenger, particular preference is given to using 1 to 1.5 equivalents. Preference is given to using 0.9 to 3 equivalents of the compounds of the formula (VII), based on compounds of the formula (VI), particular preference to 1 to 2.5 equivalents. The reaction is preferably effected at temperatures of 20 to 80° C., more preferably at temperatures of 30 to 50° C. Preference is given to carrying out the reaction under inert gas, for example nitrogen or argon. The workup is preferably effected chromatographically, more preferably by column chromatography on silica gel.

The scope of the invention also encompasses compounds of the formula (VII) where

R¹, R³ and R⁴ are each hydrogen, and R² is halogen, C₁-C₄-haloalkyl, C₁-C₄-alkoxy, C₆-C₁₀-aryloxy, cyano, C₁-C₄-alkoxycarbonyl, C₆-C₁₀-aryl-oxycarbonyl or aliphatic or aromatic C₁-C₁₀-acyloxy, and

R¹, R² and R⁴ are each hydrogen, and R³ is halogen, C₁-C₄-haloalkyl, C₁-C₄-alkoxy, C₆-C₁₀-aryloxy, cyano, C₁-C₄-alkoxycarbonyl, C₆-C₁₀-aryl-oxycarbonyl or aliphatic or aromatic C₁-C₁₀-acyloxy, and

R⁵ is hydrogen or a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group,

which may be used as intermediates for preparing the compounds of the formula (I) according to the invention where the R¹-R⁵ radicals are each as defined above.

The compounds (VII) according to the invention are preferably prepared by converting compounds of the formula (XI) in a Wittig reaction, as described, for example, in Maryanoff et al., Chem. Rev. 89, 1989, 863-927. To obtain the compounds of the formula (XI), numerous routes are conceivable and disclosed in the literature. Preference is given to starting from phenols of the formula (VI) which are converted to compounds of the formula (X) using alkylating reagents of the formula (IX) where R⁵ is as defined above and Y is a leaving group (see scheme). These may subsequently be converted to the corresponding compounds of the formula (XI) by literature methods, as described, for example, in J. Chem. Soc., Perkin Trans. 2, 1999, 1211-1218.

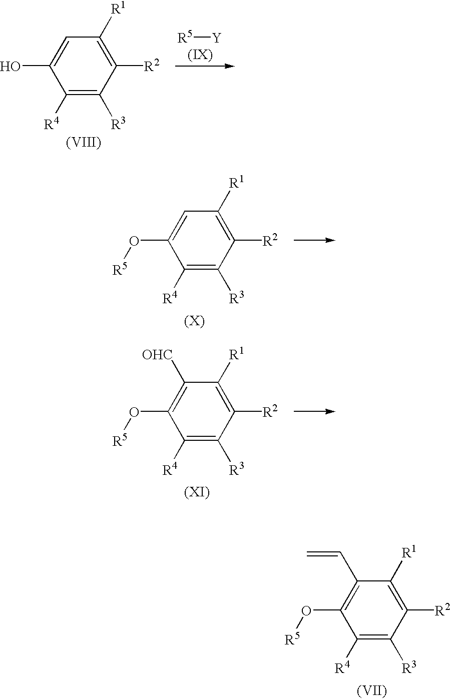

Variants which are likewise preferred for obtaining the compounds of the formula (XI) are on the one hand the conversion of phenols of the formula (VIII) to the corresponding o-aldehydes and the alkylation of these compounds to compounds of the formula (XI), and on the other hand the alkylation of salicylic acid derivatives (XII), reduction of the compounds (XIII) to compounds of the formula (XIV) and subsequent oxidation to compounds of the formula (XI) by literature methods.

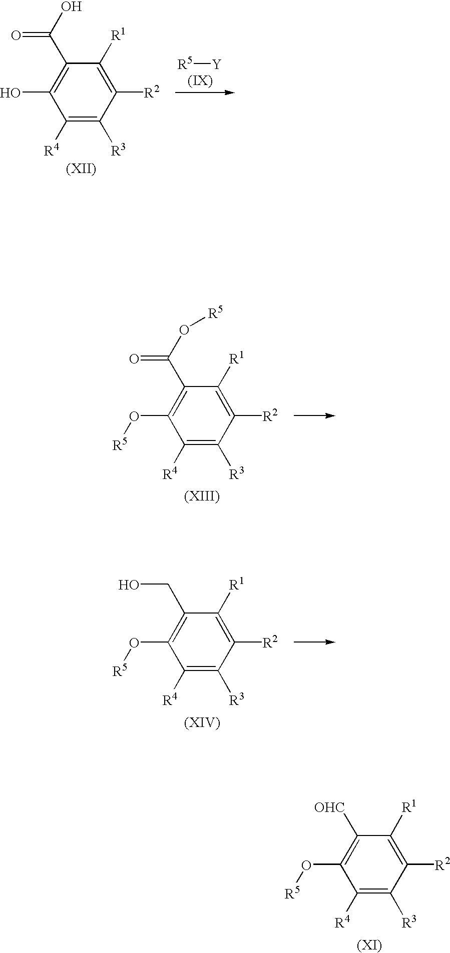

The compounds of the formula (VII) according to the invention may be used as ligands for preparing transition metal complexes, preferably for preparing transition metal complexes of the formula (I).

The compounds of the formula (I) according to the invention may be used as catalysts in chemical reactions, and preference is given to using them as catalysts in metathesis reactions, for example cross-metatheses, ring-closing metatheses and ring-opening metathesis polymerizations, optionally with subsequent cross-metathesis. Their very high activities in ring-closing metatheses are demonstrated with the aid of numerous examples of different substrates and also in comparison to existing systems. The ring-closing metatheses exhibit quantitative conversions even after only a few minutes. When used as ring-closing metathesis catalysts, the compounds of the formula (I) according to the invention lead, even at low temperatures (preferably between −10° C. and +30° C.) after a few hours virtually to quantitative yields, whereas catalysts known from the literature under comparable reaction conditions provide conversions of only ≦25% at distinctly longer reaction times.

### EXAMPLES

### Example 1

Synthesis of 2,3-diisopropoxystyrene

a) Synthesis of 2,3-diisopropoxybenzaldehyde

8.0 g (57.9 mmol) of K₂CO₃ and 8.2 g (49.2 mmol) of potassium iodide were added to a solution of 2.0 g (14.5 mmol) of 2,3-dihydroxybenzaldehyde in 50 ml of dimethylformamide, and the reaction mixture was heated to 50° C. 5.4 ml (49.2 mmol) of isopropyl bromide were slowly added dropwise and the mixture was stirred at 50° C. for a further 12 h. After the end of the reaction, the solids were filtered off and the organic phase was washed with saturated NH₄Cl solution and saturated NaCl solution, then dried over MgSO₄ and filtered, and the solvent was removed under reduced pressure. The residue was purified by column chromatography on silica gel (hexane:methyl tert-butyl ether 9:1). 2,3-Diisopropoxybenzaldehyde was obtained in a 94% yield.

¹H NMR (500 MHz, CDCl₃) δ=1.33 (d, J 6.1 Hz, 6H), 1.37 (d, J 6.1 Hz, 6H), 4.57 (septet, J 6.1 Hz, 1H), 4.66 (septet, J 6.1 Hz, 1H), 7.07 (dd, J 7.9 Hz, 1H), 7.13 (dd, J 7.6 Hz, J 1.4 Hz, 1H), 7.42 (dd, J 7.6 Hz, J 1.4 Hz, 1H), 10.45 (s, 1H) ppm.

b) Synthesis of 2,3-diisopropoxystyrene

120 ml of diethyl ether were added at 0° C. to a mixture of 4.5 g (40.5 mmol) of t-BuOK and 14.5 g (40.5 mmol) of Ph₃PCH₃Br. This suspension was stirred at 0° C. for 30 min and a solution of 3.0 g (13.5 mmol) of 2,3-diisopropoxybenzaldehyde was slowly added dropwise. The reaction mixture was stirred at 0° C. for a further hour, the solids were filtered off, the organic phase was washed with saturated NH₄Cl solution and saturated NaCl solution, then dried over MgSO₄ and filtered, and the solvent was removed under reduced pressure. The residue was purified by column chromatography on silica gel (hexane). 2,3-Diisopropoxystyrene was obtained in a 70% yield.

¹H NMR (500 MHz, CDCl₃) δ=1.30 (s, 3H), 1.31 (s, 3H), 1.37 (s, 3H), 1.50 (s, 3H), 4.48 (septet, J 6.1 Hz, 1H), 4.54 (septet, J 6.1 Hz, 1H), 5.25 (d, J 11 Hz, 1H), 5.71 (d, 17.8 Hz, 1H), 6.83 (d, 7.9 Hz, 1H), 6.83 (dd, J 7.9 Hz, 1H), 6.98 (dd, J 7.9 Hz, 1H), 7.13 (dd, J 11 Hz, J 17.8 Hz, 1H), 7.15 (d, J 7.9 Hz, 1H) ppm.

### Example 2

Synthesis of a Ruthenium Compound with 2,3-diisopropoxystyrene

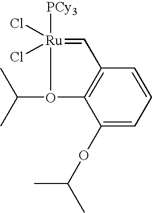

First 0.24 mmol of copper(I) chloride and then 0.24 mmol of bis(tricyclohexyl-phosphine)[benzylidene]ruthenium(IV) dichloride were added to a solution of 0.48 mmol of 2,3-diisopropoxystyrene in 20 ml of dichloromethane. After stirring at rooms temperature (23° C.) for 15 min, the reaction solution was concentrated under reduced pressure. The residue was taken up in very little dichloromethane and filtered through glass wool in a Pasteur pipette. The filtrate was concentrated again under reduced pressure and the residue chromatographed on silica gel (1:1 hexane/methylene chloride). The desired compound was isolated in a 36% yield.

¹H NMR (500 MHz, CDCl₃) δ=1.20-2.40 (m, 33H), 1.38 (d, J 6 Hz, 6H), 1.76 (d, J 6 Hz, 6H), 4.58 (septet, J 6 Hz, 1H), 6.24 (septet, J 6 Hz, 1H), 7.00 (dd, J 7.6 Hz, 1H), 7.17 (d, J 8 Hz, 1H), 7.25 (d, J 6 Hz, 1H), 17.42 (d, J 4 Hz, 1H) ppm.

### Example 3

Synthesis of 2-isopropoxy-3-methoxystyrene

a) Synthesis of 2-isopropoxy-3-methoxybenzaldehyde

18.0 g (131.4 mmol) of K₂CO₃ and 40 g (328.5 mmol) of isopropyl bromide were added to a solution of 10.0 g (65.7 mmol) of 2-hydroxy-3-methoxybenzaldehyde in 66 ml of dimethylformamide. The reaction mixture was stirred at 60° C. for 12 h. After cooling to room temperature (23° C.), the reaction mixture was added to a mixture of 100 ml of H₂O and 100 ml of saturated NH₄Cl solution and extracted using methyl tert-butyl ether. The organic phase was washed with H₂O and saturated NaCl solution, then dried over Na₂SO₄ and filtered, and the solvent was removed under reduced pressure. 2-Isopropoxy-3-methoxybenzaldehyde was obtained in an 80% yield.

¹H NMR (250 MHz, CDCl₃) δ=1.34 (d, J 6 Hz, 6H), 3.88 (s, 3H), 4.62 (septet, J 6 Hz, 1H), 7.12 (dd, J 3 Hz, 2H), 7.44 (dd, J 6 Hz, J 3 Hz, 1H), 10.46 (s, 1H) ppm.

b) Synthesis of 2-isopropoxy-3-methoxystyrene

26.5 g (74.1 mmol) of Ph₃PCH₃Br were initially charged in 240 ml of tetrahydrofuran and admixed with 4.5 g (40.5 mmol) of t-BuOK. This suspension was stirred at room temperature (23° C.) for 2 h, then cooled to −20° C., and a solution of 12.0 g (61.8 mmol) of 2-isopropoxy-3-methoxybenzaldehyde in 80 ml of tetrahydrofuran was slowly added dropwise. The reaction mixture was heated to room temperature (23° C.) and stirred for a further two hours. The reaction mixture was added to 500 ml of H₂O and extracted using methyl tert-butyl ether, the organic phase was washed with saturated NaCl solution, then dried over Na₂SO₄ and filtered, and the solvent was removed under reduced pressure. The residue was purified by column chromatography on silica gel (20:1 hexane:methyl tert-butyl ether). 2-Isopropoxy-3-methoxystyrene was obtained in a 90% yield.

¹H NMR (250 MHz, CDCl₃) δ=1.30 (d, J 6 Hz, 6H), 3.48 (s, 3H), 4.42 (septet, J 6 Hz, 1H), 5.26 (dd, J 11 Hz, J 1.4 Hz, 1H), 5.70 (dd, J 18 Hz, J 1.4 Hz, 1H), 6.82 (dd, J 8 Hz, J 1.4Hz, 1H), 7.02 (dd, J 8,Hz, 1H), 7.06 (d, J 8 Hz, 1H), 7.16 (dd, 8 Hz, J 1.4 Hz, 1H) ppm.

### Example 4

Synthesis of a Ruthenium Compound Using 2-isopropoxy-3-methoxystyrene

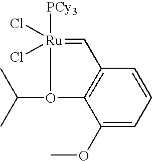

First 20 mg (0.2 mmol) of copper(I) chloride and then 150 mg (0.19 mmol) of bis(tricyclohexylphosphine) [benzylidene]ruthenium(IV) dichloride were added to a solution of 82 mg (0.37 mmol) of 2-isopropoxy-3-methoxystyrene in 10 ml of dichloromethane. After stirring at room temperature (23° C.) for 12 hours, the reaction solution was concentrated under reduced pressure. The residue was taken up in very little dichloromethane and filtered through glass wool in a Pasteur pipette. The filtrate was concentrated again under reduced pressure and the residue chromatographed on silica gel (1:1 hexane/methylene chloride). The desired compound was isolated in a 60% yield.

¹H NMR (250 MHz, CDCl₃) δ=1.00-2.40 (m, 33H), 1.26 (d, J 6 Hz, 6H), 1.77 (d, J 6 Hz, 6H), 3.88 (s, 3H), 6.14 (septet, J 6 Hz, 1H), 7.04 (m, 2H), 7.23 (d, J 6 Hz, 1H), 17.42 (d, J 4 Hz, 1H) ppm.

### Example 5

Synthesis of 2,5-diisopropoxystyrene

a) Synthesis of 2,5-diisopropoxybenzaldehyde

8.76 g (63.35 mmol) of K₂CO₃, 0.51 g (1.38 mmol) of tetrabutylammonium iodide and 6.23 g (50.68 mmol) of isopropyl bromide were added to a solution of 1.75 g (12.67 mmol) of 2,5-dihydroxybenzaldehyde in 30 ml of dimethylformamide, and the reaction mixture was stirred at 50° C. for 12 h. After the end of the reaction, the mixture was washed with saturated NaCl solution and H₂O, then dried over MgSO₄ and filtered, and the solvent was removed under reduced pressure. The residue was purified by column chromatography on silica gel (9:1 hexane:methyl tert-butyl ether). 2,5-Diisopropoxybenzaldehyde was obtained in a 76% yield.

¹H NMR (500 MHz, CDCl₃) δ=1.31 (d, J 6 Hz, 6H), 1.37 (d, J 6 Hz, 6H), 4.50 (septet, J 6 Hz, 1H), 4.56 (septet, J 6 Hz, 1H), 6.93 (d, J 10 Hz, 1H), 7.08 (d, J 10 Hz, 1H), 7.35,(s, 1H), 10.45 (s, 1H) ppm.

b) Synthesis of 2,5-diisopropoxystyrene

1.77 g (4.96 mmol) of Ph₃PCH₃Br were added to 10 ml of tetrahydrofuran, the mixture was cooled to 0° C. and 3.1 ml (4.54 mmol) of a 1.5 M solution of butyllithium in tetrahydrofuran were slowly added dropwise. The mixture was stirred at 0° C. for 10 min and a solution of 1.0 g (4.54 mmol) of 2,5-diisopropoxy-benzaldehyde in 2 ml of tetrahydrofuran was added dropwise. The suspension was subsequently stirred at room temperature (23° C.) for one hour and stirred under reflux for a further hour. After cooling to room temperature (23° C.), H₂O was added, extraction was effected using methyl tert-butyl ether, the organic phase was dried over MgSO₄ and filtered, and the solvent was removed under reduced pressure. The residue was purified by column chromatography on silica gel (4:1 hexane:methylene chloride). 2,5-Diisopropoxystyrene was obtained in an 81% yield.

¹H NMR (500 MHz, CDCl₃) δ=1.25 (d, J 6 Hz, 6H), 1.27 (d, J 6 Hz, 6H), 4.32 (septet, J 6 Hz, 1H), 4.40 (septet, J 6 Hz, 1H), 5.17 (dd, J 11 Hz, J 1.4 Hz, 1H), 5.64 (dd, J 17 Hz, J 1.4 Hz, H), 6.70 (dd, J 9 Hz, J 3 Hz, 1H), 6.76 (d, J 9 Hz, 1H), 6.97 (dd, J 17 Hz, J 11 Hz, 1H), 6.98 (d, J 3 Hz, 1H) ppm.

### Example 6

Synthesis of a Ruthenium Compound with 2,5-diisopropoxystyrene

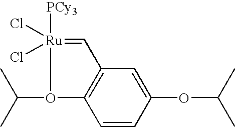

0.61 mmol of bis(tricyclohexylphosphine)[benzylidene]ruthenium(IV) dichloride was added to a solution of 0.73 mmol of 2,5-diisopropoxystyrene in 12 ml of dichloromethane. After stirring at room temperature (23° C.) for 48 h, the reaction solution was concentrated under reduced pressure. The residue was taken up in very little dichloromethane and filtered through glass wool in a Pasteur pipette. The filtrate was concentrated again under reduced pressure and the residue chromatographed on silica gel (1:1 hexane/methylene chloride). The desired compound was isolated in a 66% yield.

¹H NMR (500 MHz, CDCl₃) δ=1.20-2.40 (m, 33H), 1.35 (d, J 6 Hz, 6H), 1.78 (d, J 6 Hz, 6H), 4.50 (septet, J 6 Hz, 1H), 5.19 (septet, J 6 Hz, 1H), 6.96 (d, J 9 Hz, 1H), 7.17 (d, J 3 Hz, 1H), 7.19 (s, 1H), 17.33 (d, J 4 Hz, 1H) ppm.

### Example 7

Synthesis of 2-fluoro-5-isopropoxystyrene

a) Synthesis of 2-fluoro-5-isopropoxybenzaldehyde

6.1 g (32.6 mmol) of MnO₂ are added with stirring to a solution of 1.2 g (6.5 mmol) of (2-fluoro-5-isopropoxyphenyl)methanol in 12 ml of diethyl ether. The reaction mixture is stirred at 23° C. for 4 h. After the end of the reaction, the mixture is filtered through a little celite and washed through with diethyl ether. After removing the solvent, 1.2 g (90% yield) of 2-fluoro-5-isopropoxybenzaldehyde are obtained as a colourless oil.

¹H NMR (200 MHz, CDCl₃): δ=1.42 (6 H, d, J 6 Hz), 4.62 (1 H, quintett, J 6 Hz), 6.96 (1 H, dd, J 10 Hz, J 6 Hz), 7.07-7.14 (1 H, m), 7.48 (1 H, dd, J 8 Hz, J 4 Hz), 10.40 (1 H, d, J 4 Hz) ppm.

b) Synthesis of 2-fluoro-5-isopropoxystyrene

1.4 g (12.7 mmol) of t-BuOK are added at 0° C. with stirring to a suspension of 4.5 g (12.7 mmol) of Ph₃PCH₃Br in 36 ml of diethyl ether. The reaction mixture is stirred at 0° C. for 10 mm and then a solution of 1.2 g (6.4 mmol) of 2-fluoro-5-isopropoxybenzaldehyde in 24 ml of diethyl ether is slowly added dropwise. The mixture is stirred at 0° C. for a further 10 min, then heated to room temperature (23° C.) and then quenched using saturated NH₄Cl solution. The mixture is then extracted using diethyl ether and the organic phase is washed with water and saturated NaCl solution. After drying over MgSO₄, the solvent is distilled off and 1.0 g (89% yield) of 2-fluoro-5-isopropoxystyrene is obtained as a colourless oil.

¹H NMR (500 MHz, CDCl₃): δ=1.33 (6 H, d, J 6 Hz), 4.43 (1 H, quintett, J 6 Hz), 5.28(1 H, d, J 11 Hz), 5.71 (1 H, d, J 17.7 Hz), 6.83 (1 H, dd, J 4.7 Hz, J 8.8 Hz), 6.88 (1 H, ddd, J 1.6 Hz, J 7.8 Hz), 7.02 (1 H, dd, J 17.7 Hz, J 11.1 Hz), 7.18 (1 H, dd, J 9.5 Hz, J 2.5 Hz) ppm.

### Example 8

Synthesis of a Ruthenium Compound Using 2,-fluoro-5-isopropoxystyrene

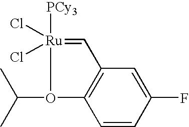

A solution of 59 mg (0.32 mmol) of 2-fluoro-5-isopropoxystyrene in 3 ml of CH₂Cl₂ is added to a solution of 133 mg (0.32 mmol) of bis(tricyclohexylphosphine)[benzylidene]ruthenium(IV) dichloride and 16 mg (0.16 mmol) of CuCl in 10 ml of CH₂Cl₂. The reaction mixture is stirred at 25° C. for 12 h. After the end of the reaction, the solvent is removed on a rotary evaporator and the residue is dissolved in a little CH₂Cl₂ and this solution is filtered through a little cotton wool. The solvent is removed again on a rotary evaporator and the residue is chromatographed on SiO₂ (9:1 cyclohexane:ethyl acetate). 90 mg (45% yield) of the desired compound are obtained.

¹H NMR (500 MHz, C₆D₆) δ=1.11-2.42 (m, 33H), 1.69 (d, J 6 Hz, 6H), 4.59 (quintett, J 6 Hz, 1H), 6.26 (dd, J 9 Hz, J 4 Hz, 1H), 6.89-6.91 (m, 1H), 6.99 (dd, J 8 Hz, J 4 Hz, 1H), 17.09 (d, J 4 Hz, 1H) ppm.

### Example 9

RCM, Using the Compound of Example 2 as Catalyst

A 0.01 M solution of N,N-bisallyltosylamide in dichloromethane was admixed at room temperature with 1 mol % of the compound of Example 2. The conversion of the reaction as a function of time was determined by means of HPLC at room temperature via the reactant/product ratio. (HPLC conditions: RP-7 column (4 mm), eluent: 8:1 methanol:H₂O, wavelength: 254 nm, retention time: reactant 4.42 min, product 3.15 min). The results are listed in Table 1.

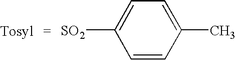

In a similar manner, the conversion was determined when using the catalyst of the formula (A) (Hoveyda et al., J. Am. Chem. Soc. 1999, 121, 791-799)

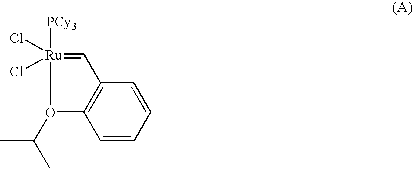

where

PCy₃ is a tricyclohexylphosphine radical. The results are listed in Table 1.

### Example 10

RCM, Using the Compound of Example 4 as Catalyst

A 0.01 M solution of N,N-bisallyltosylamide in dichloromethane was admixed at room temperature with 1 mol % of the compound of Example 4. The conversion of the reaction as a function of time was determined by means of HPLC at room temperature via the reactant/product ratio. (HPLC conditions: RP-7 column (4 mm), eluent: 8:1 methanol:H₂O, wavelength: 254 nm, retention time: reactant, 4.42 min, product 3.15 min). The results are listed in Table 1.

In a similar manner, the conversion was determined when using 1 mol % of the catalyst of formula (A). The results are listed in Table 1.

### Example 11

RCM, Using the Compound of Example 6 as Catalyst

A 0.01 M solution of N,N-bisallyltosylamide in dichloromethane was admixed at room temperature with 1 mol % of the compound of Example 6. The conversion of the reaction as a function of time was determined by means of HPLC at room temperature via the reactant/product ratio. (HPLC conditions: RP-7 column (4 mm), eluent: 8:1 methanol:H₂O, wavelength: 254 nm, retention time: reactant 4.42 min, product 3.15 min). The results are listed in Table 1.

In a similar manner, the conversion was determined when using 1 mol % of the catalyst of formula (A). The results are listed in Table 1.

### Example 12

RCM, Using the Compound of Example 8 as Catalyst

A 0.01 M solution of N,N-bisallyltosylamide in dichloromethane was admixed at room temperature with 1 mol % of the compound of Example 8. The conversion of the reaction as a function of time was determined by means of HPLC at room temperature via the reactant/product ratio. (HPLC conditions: RP-7 column (4 mm), eluent: 8:1 methanol:H₂O, wavelength: 254 nm, retention time: reactant 4.42 min, product 3.15 min). The results are listed in Table 1.

In a similar manner, the conversion was determined when using 1 mol % of the catalyst of formula (A). The results are listed in Table 1.

TABLE 1

| | Conversion (%) | | | | |
|---|---|---|---|---|---|
| | | Compound | Compound | Compound | Compound |
| Time | Catalyst | of | of | of | of |
| (min) | (A) | Example 2 | Example 4 | Example 6 | Example 8 |
| 3 | 5 | 8 | 7 | 0 | 3 |
| 10 | 7 | 45 | — | 1 | — |
| 18 | 10 | 74 | 69 | 24 | — |
| 25 | 17 | 83 | 79 | 40 | 34 |
| 33 | 26 | 86 | 84 | 57 | — |
| 48 | 47 | — | 86 | 76 | 81 |
| 62 | 64 | 90 | 88 | — | 89 |
| 90 | 76 | — | — | 88 | — |
| 107 | 82 | 91 | — | 89 | 93 |
| 125 | 84 | 91 | — | — | — |
| 140 | 85 | 92 | 88 | 89 | 92 |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Compounds of the formula (I) 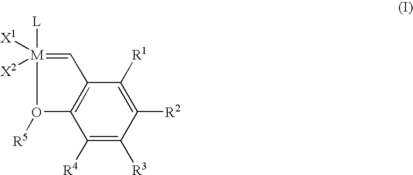
characterized in that
M is a transition metal of the 8th transition group of the Periodic Table,
X¹ and X² are the same or different and are each an anionic ligand,
R¹, R², R³ and R⁴ are the same or different and are each hydrogen, with the proviso that at least one radical R¹ to R⁴ is different from hydrogen, or are each cyclic, straight-chain or branched alkyl radicals having 1 to 50 carbon atoms or aryl radicals having 6 to 30 carbon atoms, at least one hydrogen atom in the radicals mentioned optionally being replaced by an alkyl group or a functional group, and at least one of the radicals R¹ to R⁴ is halogen, C₁-C₄-haloalkyl, C₁-C₄-alkoxy, C₆-C₁₀-aryloxy, cyano, C₁-C₄-alkoxy-carbonyl, C₆-C₁₀-aryloxycarbonyl or aliphatic or aromatic C₁-C₁₀-acyloxy, and/or
R¹ and R² or R² and R³ or R³ and R⁴ or R⁴ and R⁵ are part of a cyclic system which consists of a carbon framework having 3 to 20 carbon atoms, not including the carbon atoms in formula (I), at least one hydrogen atom optionally being replaced by an alkyl group or a functional group, and/or at least one carbon atom of the cycle optionally being replaced by a heteroatom from the group of S, P, O and N, and
R⁵ is hydrogen or a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, and R² and R³ may not be part of a cyclic, aromatic system having 4 carbon atoms, not including the carbon atoms in formula (I), when R⁵ is at the same time methyl, and
L is a neutral two-electron donor from the group of amines, imines, phosphines, phosphites, stibines, arsines, CO, carbonyl compounds, nitrites, alcohols, thiols, ethers and thioethers.

2. Compounds according to claim 1, characterized in that
M is ruthenium or osmium,
X¹ and X² are the same or different and are each an anionic ligand from the group of halides, pseudohalides, hydroxides, alkoxides, carboxylates and sulphonates,
R¹, R², R³ and R⁴ are the same or different and are each hydrogen, with the proviso that at least one radical R¹ to R⁴ is different to hydrogen, or are each cyclic, straight-chain or branched alkyl radicals having 1 to 20 carbon atoms or aryl radicals having 6 to 20 carbon atoms, at least one hydrogen atom in the alkyl and aryl radicals mentioned optionally being replaced by a functional group, and at least one of the radicals R¹ to R⁴ is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or
R¹, R² and R³ are each hydrogen and R⁴ is a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or
R², R³ and R⁴ are each hydrogen and R¹ is a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxy-carbonyl or aliphatic or aromatic C₁-C₁₀-acyloxy, or
R¹, R³ and R⁴ are each hydrogen and R² is a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or
R¹, R² and R⁴ are each hydrogen and R³ is a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or
R¹ and R⁴ are the same or different and are each hydrogen or an aryl radical having 6 to 20 carbon atoms, at least one hydrogen atom in the aryl radical optionally being replaced by an alkyl group or a functional group, or are each halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy and R² and R³ are part of a cyclic aromatic system having 4 to 14 carbon atoms, not including the carbon atoms in formula (I) of claim 1, at least one hydrogen atom optionally being replaced by an alkyl group or a functional group, or
R¹ and R² are the same or different and are each hydrogen or an aryl radical having 6 to 20 carbon atoms, at least one hydrogen atom in the aryl radical optionally being replaced by an alkyl group or a functional group, or are each halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy and R³ and R⁴ are part of a cyclic aromatic system having 4 to 14 carbon atoms, not including the carbon atoms in formula (I) of claim 1, at least one hydrogen atom optionally being replaced by an alkyl group or a functional group,
R⁵ is a straight-chain or branched alkyl radical having 1 to 20 carbon atoms, and R² and R³ may not be part of a cyclic, aromatic system having 4 carbon atoms, not including the carbon atoms in formula (I), when R⁵ is at the same time methyl, and
L is as defined in claim 1.

3. Compounds according to claim 1, characterized in that
L is a phosphine ligand PR⁷R⁸R⁹, with the proviso that R⁷, R⁸ and R⁹ may be the same or different and are each cyclic, straight-chain or branched alkyl radicals having 1 to 10 carbon atoms or aryl radicals having 6 to 14 carbon atoms, at least one hydrogen atom in the alkyl or aryl radicals mentioned optionally being replaced by an alkyl group or a functional group, and
M, X¹, X², R¹ to R⁵ are as defined in claim 1.

4. Compounds according to claim 1, characterized in that
M is ruthenium,
X¹ and X² are the same and are each an anionic ligand from the group of halides and pseudohalides,
R¹, R², R³ and R⁴ are the same or different and are each hydrogen, with the proviso that at least one radical R¹ to R⁴ is different to hydrogen, or are each cyclic, straight-chain or branched allyl radicals having 1 to 10 carbon atoms or aryl radicals having 6 to 14 carbon atoms, at least one hydrogen atom in the alkyl or aryl radicals mentioned optionally being replaced by an alkyl group or a functional group, and at least one of the radicals R¹ to R⁴ is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic aromatic C₁-C₁₀-acyloxy, or
R¹, R² and R³ are each hydrogen and R⁴ is an aryl radical having 6 to 14 carbon atoms, at least one hydrogen atom in the aryl radical optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or
R², R³, and R⁴ are each hydrogen and R¹ is a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or
R¹, R³ and R⁴ are each hydrogen and R² is a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or
R¹, R² and R⁴ are each hydrogen and R³ is a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, or
R¹ is hydrogen or halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy and R⁴ is hydrogen or an aryl radical having 6 to 14 carbon atoms, at least one hydrogen atom in the aryl radical optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxy-carbonyl or aliphatic or aromatic C₁-C₁₀-acyloxy, and R² and R³ are part of a cyclic aromatic system having 4 to 8 carbon atoms, not including the carbon atoms in formula (I) of claim 1, and at least one hydrogen atom optionally being replaced by an alkyl group or a functional group, or
R¹ is hydrogen or halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy and R² is hydrogen or an aryl radical having 6 to 14 carbon atoms, at least one hydrogen atom in the aryl radical optionally being replaced by an alkyl group or a functional group, or is halogen, C₁-C₄-haloalkyl, C₁-C₄-alkoxy, C₆-C₁₀-aryloxy, cyano, C₁-C₄-alkoxycarbonyl, C₆-C₁₀-aryloxy-carbonyl or aliphatic or aromatic C₁-C₁₀-acyloxy, and R³ and R⁴ are part of a cyclic aromatic system having 4 to 8 carbon atoms, not including the carbon atoms in formula (I) of claim 1, and at least one hydrogen atom optionally being replaced by an alkyl group or a functional group,
R⁵ is a branched alkyl radical having 3 to 8 carbon atoms, and
L is a phosphine ligand PR⁷R⁸R⁹, with the proviso that R⁷, R⁸ and R⁹ are the same and are each methyl, ethyl, cyclopentyl, cyclohexyl or phenyl.

5. Compounds according to claim 1, characterized in that
M is ruthenium,
X¹ and X² are the same and are each halide,
R¹, R² and R³ are each hydrogen and R⁴ is a phenyl or naphthyl radical, at least one hydrogen atom optionally being replaced by an alkyl group or a functional group, or is a radical from the group of F, Cl, Br, trifluoromethyl, methoxy, ethoxy, isopropoxy, tert-butoxy, phenoxy, cyano, methoxycarbonyl, ethoxycarbonyl, tert-butoxycarbonyl, acetoxy, propionyloxy and pivaloyloxy, or
R¹, R² and R⁴ are each hydrogen and R³ is a radical from the group of F, Cl, Br, trifluoromethyl, methoxy, ethoxy, isopropoxy, tert-butoxy, phenoxy, cyano, methoxycarbonyl, ethoxycarbonyl, tert-butoxy-carbonyl, acetoxy, propionyloxy and pivaloyloxy, or
R¹, R³, and R⁴ are each hydrogen and R² is a radical from the group of F, Cl, Br, trifluoromethyl, methoxy, ethoxy, isopropoxy, tert-butoxy, phenoxy, cyano, methoxycarbonyl, ethoxycarbonyl, tert-butoxy-carbonyl, acetoxy, propionyloxy and pivaloyloxy, or
R², R³ and R⁴ are each hydrogen and R¹ is a radical from the group of F, Cl, Br, trifluoromethyl, methoxy, ethoxy, isopropoxy, tert-butoxy, phenoxy, cyano, methoxycarbonyl, ethoxycarbonyl, tert-butoxy-carbonyl, acetoxy, propionyloxy and pivaloyloxy, or
R¹ is hydrogen or a radical from the group of F, Cl, Br, trifluoromethyl, methoxy, ethoxy, isopropoxy, tert-butoxy, phenoxy, cyano, methoxycarbonyl, ethoxycarbonyl, tert-butoxycarbonyl, acetoxy, propionyloxy and pivaloyloxy and R⁴ is hydrogen or phenyl or naphthyl, at least one hydrogen atom optionally being replaced by an alkyl group or a functional group, or is a radical from the group of F, Cl, Br, trifluoromethyl, methoxy, ethoxy, isopropoxy, tert-butoxy, phenoxy, cyano, methoxycarbonyl, ethoxycarbonyl, tert-butoxycarbonyl, acetoxy, propionyloxy and pivaloyloxy and R² and R³ are part of a cyclic aromatic system having 4 to 8 carbon atoms, not including the carbon atoms in formula (I), and at least one hydrogen atom optionally being replaced by an alkyl group or a functional group, or
R¹ is hydrogen or a radical from the group of F, Cl, Br, trifluoromethyl, methoxy, ethoxy, isopropoxy, tert-butoxy, phenoxy, cyano, methoxycarbonyl, ethoxycarbonyl, tert-butoxycarbonyl, acetoxy, propionyloxy and pivaloyloxy and R² is hydrogen or phenyl or naphthyl, at least one hydrogen atom optionally being replaced by an alkyl group or a functional group, or is a radical from the group of F, Cl, Br, trifluoromethyl, methoxy, ethoxy, isopropoxy, tert-butoxy, phenoxy, cyano, methoxycarbonyl, ethoxycarbonyl, tert-butoxycarbonyl, acetoxy, propionyloxy and pivaloyloxy and R³ and R⁴ are part of a cyclic aromatic system having 4 to 8 carbon atoms, not including the carbon atoms in formula (I), and at least one hydrogen atom optionally being replaced by an alkyl group or a functional group,
R⁵ is a branched alkyl radical from the group of isopropyl, isobutyl, sec-butyl, tert-butyl, branched pentyl, branched hexyl, and
L is a phosphine PR⁷R⁸R⁹, with the proviso that R⁷, R⁸ and R⁹ are the same and are each methyl, ethyl, cyclopentyl, cyclohexyl or phenyl.

6. Process for preparing compounds of the formula (I) according to claim 1, characterized in that the phosphine ligand PZ₃ in compounds of the formula (VI) 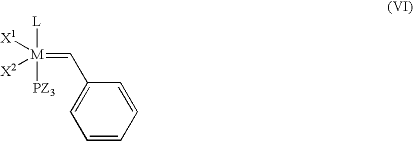
where
L is as defined in claim 1,
PZ₃ is a phosphine ligand, in particular trimethylphosphine, triethyl-phosphine, tricyclopentylphosphine, tricyclohexylphosphine or triphenylphosphine,
M, X¹ and X² are each as defined in claim 1 is exchanged by ligands of the formula (VII) 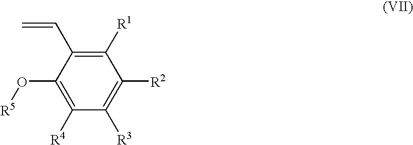
where
R¹ to R⁵ are each as defined in claim 1.

7. Process according to claim 6, characterized in that the reaction takes place in the presence of compounds which are capable of scavenging phosphines.

8. Compounds of the formula (VII) 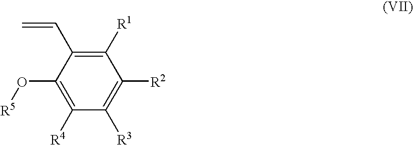
characterized in that
R¹, R³ and R⁴ are each hydrogen, and R² is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, and
R¹, R² and R⁴ are each hydrogen, and R³ is halogen, C₁-C₄-haloalkyl, C₁-C₄-alkoxy, C₆-C₁₀-aryloxy, cyano, C₁-C₄-alkoxycarbonyl, C₆-C₁₀-aryloxycarbonyl or aliphatic or aromatic C₁-C₁₀-acyloxy, and
R⁵ is hydrogen or a cyclic, straight-chain or branched alkyl radical having 1 to 20 carbon atoms or an aryl radical having 6 to 20 carbon atoms, at least one hydrogen atom in the radicals mentioned optionally being replaced by an alkyl group or a functional group.

9. Process for preparing compounds of the formula (VI) according to claim 8, comprising converting compounds of the formula (XI) 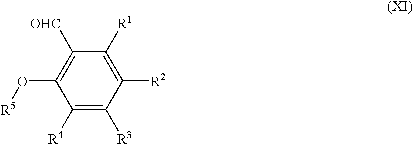
in a Wittig reaction.

10. A process for catalyzing reactions comprising providing compounds of the formula (I) according to claim 1.

11. A process for performing metathesis reaction comprising providing compounds of the formula (I) according to claim 1.

12. A process for preparing transition metal complexes comprising providing compounds of the formula (VII) according to claim 1.
